(19) United States
(12) Patent Application Publication
(10) Pub. No.: US 2023/0132938 A1
(43) Pub. Date: May 4, 2023

(54) SUNGLASSES FOR USE IN WATER ACTIVITIES

(71) Applicant: Rodrigo DANGELO, Curitiba (BR)

(72) Inventor: Rodrigo DANGELO, Curitiba (BR)

(21) Appl. No.: 17/860,597

(22) Filed: Jul. 8, 2022

(30) Foreign Application Priority Data
Oct. 29, 2021 (BR) BR 102021021834-7

(51) Int. Cl. G02C 5/00; G02C 3/00

(52) U.S. Cl. CPC G02C 5/001; G02C 3/003

(57) ABSTRACT

The present patent of invention is described as sunglasses for use in water activities which, according to its characteristics, provides sunglasses for use in water activities (1) in a specific, stand-alone structure of the universal type and based on a structuring frame (2), two sealing/protective eye cups (3) for water environments, two lenses (4) for solar environments (glare/bright light), two straps (5) to secure the glasses in place on the head, two pins (6) to hold the straps to the frame and a strap adjuster (7) integrated into a single block to attach/fix the glasses around the eyes and the head, in order to provide complete optimization of the sunglasses for use in water activities (1) in combined or stand-alone applications for protection, viewing and correction in solar and water environments, including sports practices, without any types of adjustments to the assembly.

### CROSS-REFERENCE TO RELATED APPLICATIONS

The present application claims priority benefit of Brazil Application No. BR 10 2021 021834-7, filed Oct. 29, 2021, which application is hereby incorporated herein by reference in its entirety.

### TECHNICAL FIELD

The present patent of invention relates to eyeglasses in general, more specifically to sunglasses for use in water activities which, according to the general characteristics thereof, provides, as its basic principle, the formation of sunglasses for use in water activities in a specific, stand-alone structure of universal type and based on a main frame, two sealing/protective eye cups for water environments, two viewing/protective lenses for solar environments (glare/bright light), two straps to secure the glasses in place on the head, two pins to hold the straps to the frame and a strap adjuster perfectly integrated into a single block to attach/fix the glasses around the wearer's eyes and head.

### BACKGROUND

The aim of said structure is to provide, in an extremely practical, safe and accurate manner, complete optimization of both combined or stand-alone applications of the sunglasses for eye protection and correction in solar and water environments, including sports practices, without any types of adjustments to the assembly and based on sunglasses for use in water activities of high strength, safety and versatility.

With a specific design and format and easy access for adaptation and user safety, convenience features in handling and functionality, very affordable and, due to its general characteristics and dimensions, easily adaptable to a variety of frames, lenses, solar and water environments, locations, and users in general, regardless of the characteristics that they may present.

Three basic types of eyeglasses according to their application/usage have been widely known in the current state of the art: prescription eyeglasses for eyesight correction due to some kind of visual impairment such as myopia and astigmatism; solar protection glasses for eyeball protection due to excessive sunlight; and water protection glasses—for eye protection and underwater viewing in water environments while diving or swimming.

Each of these three types of eyeglasses are provided with a proper and specific stand-alone structure directly based on the application to which they are intended by means of elements that provide a safe and comfortable application by users, the interchangeability of which is not allowed.

In an extensive review of the literature in order to provide the current state of the art of recreational and/or sports sunglasses, more specifically the integration between this and the other applications thereof, object of the present patent of invention, documents relevant to the prior art related to the specific object claimed in the present invention were not described, namely, eyeglasses incorporating in a single structure the requirements for visual correction, solar and water protection for users' eyes without any types of adjustments.

Currently, only prescription glasses and sunglasses provide perfect, safe and ergonomic integration into a single structure, with no need for accessories and adjustments, simultaneously providing eyesight correction and solar protection, while keeping high aesthetics by the use of a single lens.

Water protection glasses, or swimming goggles, require very specific structuring, mainly due to the need of full watertightness/sealing around the eyes, besides being made from rubberized material, including the lenses, and, therefore, do not allow a perfect and safe integration to the other applications. Likewise, swimming goggles do not allow the use of any kind of contemporary design, being based on large goggles for recreation or diving purposes or small goggles for swimming competitions.

In this line of action, it has become essential for eyewear users, mainly for those wearing glasses in water environments, both in social, recreational and sports activities, the structuring of a versatile, practical, safe and accurate means capable of allowing complete optimization in simultaneous correction and solar protection applications, when in water environments, that is, providing visual correction, solar protection and water protection (watertightness) in an integrated manner.

Thus, the present patent of invention is characterized by combining integrated components in a differentiated design that meets the various requirements the nature of its use demands, i.e., viewing/protection simultaneously to both solar and/or water environments. The concept ensures sunglasses for use in water activities of great efficiency, functionality, strength, durability, safety, versatility and ergonomics due to the excellent aggregate technical qualities, which provide advantages and improvements in the use of eyeglasses simultaneously in solar environments (glare/bright light) and water environments and whose general characteristics differ from the other shapes and models widely known in the current state of the art.

The present patent of invention comprises the use of modern, efficient, safe and functional sunglasses for use in water activities formed by a set of properly incorporated mechanical and aesthetic eyesight solutions, forming complete and differentiated sunglasses for use in water activities with unique design, great finish details and specific characteristics, is aesthetically pleasing and which incorporates its own specific universal type, containing an integrated and symmetrically arranged frame as a structuring element for the eyeglasses, two eyes cups as sealing/protective elements for the eye area in water environments, two lenses as viewing/protective elements in solar environments, two straps as support elements of the glasses on the head, two pins as locking elements of the straps to the frames and a strap adjuster as an adjusting element for the glasses on the head, so as to provide a unique, complete and safe assembly, whose shapes and internal and external arrangements enable perfect adaptation to a wide variety of users' eyes and heads, being specially designed for these purposes.

The overall design of the present sunglasses for use in water activities, object of the present patent of invention, is entirely based on its simple and robust structure with a minimum required number of components and extremely simple, safe and optimized operation, combined with fairly practical manufacturing procedures so as to generate practical and efficient sunglasses for use in water activities capable of being ergonomically fixed around the head and over the eye area and integrating, into a single structure, all the elements required to simultaneously provide eyesight protection, viewing and correction in solar and water environments, including sports practices, without any types of adjustments to the assembly, i.e., creating the proper conditions for eyesight correction, solar protection and water protection to the users' eyes.

In view of this and other points, a new type of contemporarily-shaped pair of glasses placed around the eye area has been developed by means of a frame, two sealing/protective eye cups for water environments and two lenses for viewing/protection/correction for solar environments, and subsequently fixed to the head area by means of two straps, two pins to hold the straps to the frame and a strap adjuster.

Following the same structural concept, the sunglasses for use in water activities provide, in a fully practical way, a series of combinations among all of its constituent parts, both regarding the design and the protective/corrective performance, according to the users' needs.

The sunglasses for use in water activities are based on the application of components and processes in a differentiated concept without, however, achieving a high degree of sophistication and complexity, thus making it possible to solve some of the main drawbacks of other shapes and models known in the state of the art and employed in combined applications for solar and water protection, as well as eyesight, which are located in an operating range in which are very common the limitations on use and application, low efficiency and performance, accidents of different severities, great wear and fragility, low durability and resistance, low versatility, high cost, general high volume and weight, low ergonomics, little flexibility and mobility and low support capacity.

### BRIEF DESCRIPTION OF THE DRAWINGS

The objectives, advantages, and other important characteristics of the utility model in question can be more easily understood when read in conjunction with the accompanying drawings, in which:

FIG. 1A is a top perspective view of the sunglasses for use in water activities.

FIG. 1B is a bottom perspective view of the sunglasses for use in water activities.

FIG. 1C is a top view of the sunglasses for use in water activities.

FIG. 1D is a bottom view of the sunglasses for use in water activities.

FIG. 1E is a right-side view of the sunglasses for use in water activities.

FIG. 1F is a left-side view of the sunglasses for use in water activities.

FIG. 1G is a front view of the sunglasses for use in water activities.

FIG. 1H is a rear view of the sunglasses for use in water activities.

FIG. 1I is an exploded top perspective view of the sunglasses for use in water activities.

FIG. 1J is an exploded bottom perspective view of the sunglasses for use in water activities.

FIG. 2A is a top perspective view of the pad of the sunglasses for use in water activities.

FIG. 2B is a bottom perspective view of the pad of the sunglasses for use in water activities.

FIG. 3 is a perspective view of the lenses of the sunglasses for use in water activities.

FIG. 4A is a rear perspective view of the frame of the sunglasses for use in water activities.

FIG. 4B is a detailed rear perspective view of the frame of the sunglasses for use in water activities.

FIG. 5 is a detailed perspective view of the strap tips of the sunglasses for use in water activities.

FIG. 6A is a detailed perspective view of the fastening of the strap to the frame of the sunglasses for use in water activities in a first fastening step.

FIG. 6B is a detailed perspective view of the fastening of the strap to the frame of the sunglasses for use in water activities in a second fastening step.

FIG. 6C is a detailed perspective view of the fastening of the strap to the frame of the sunglasses for use in water activities in a third fastening step.

FIG. 6D is a detailed perspective view of the fastening of the strap to the frame of the sunglasses for use in water activities in a fourth fastening step.

### DETAILED DESCRIPTION

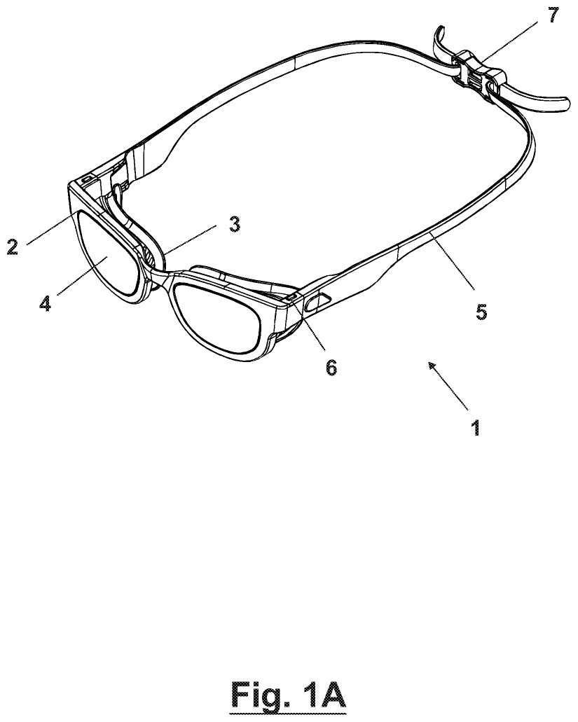

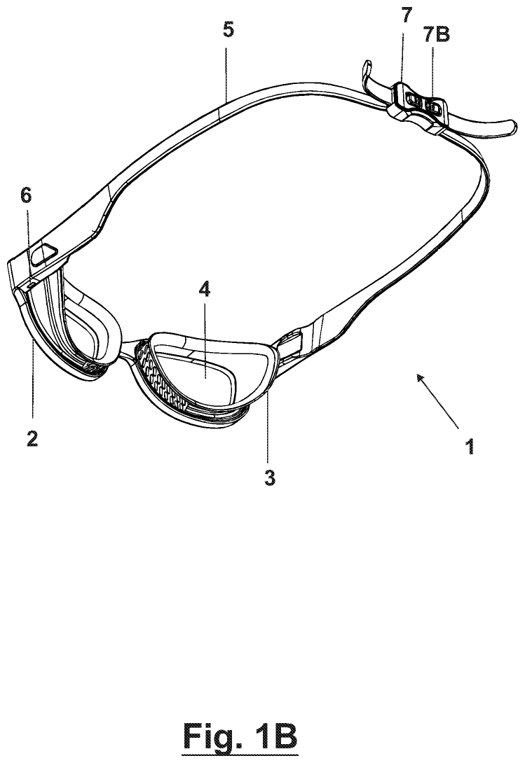

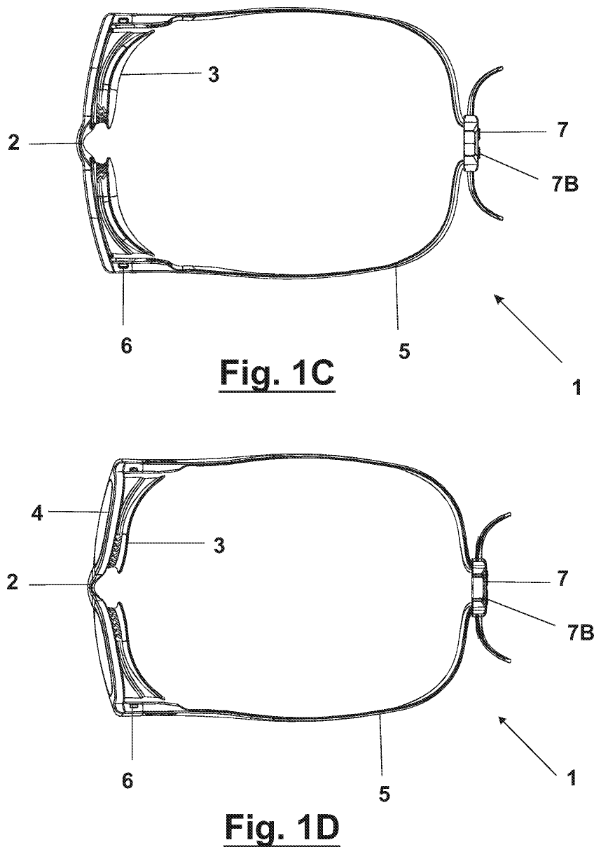

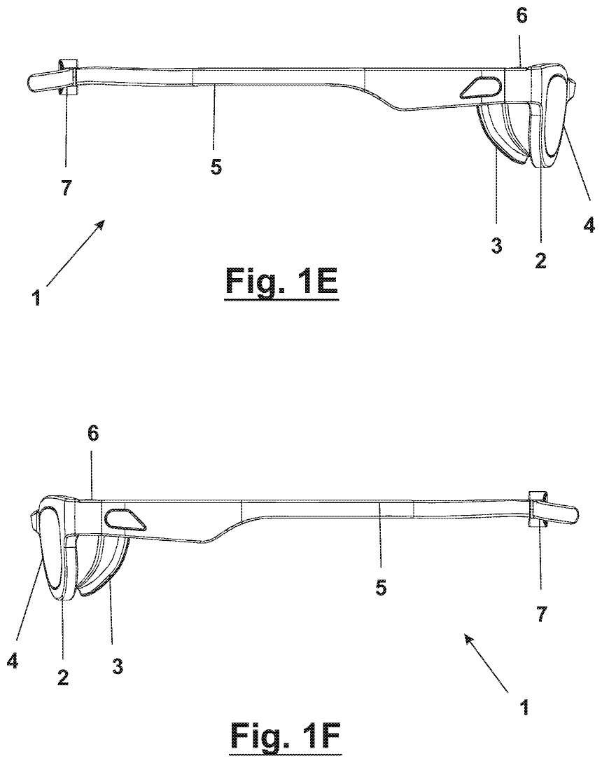

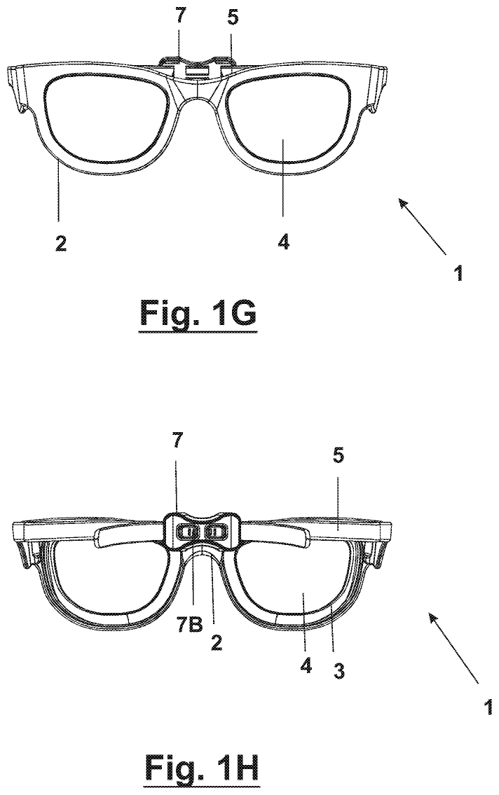

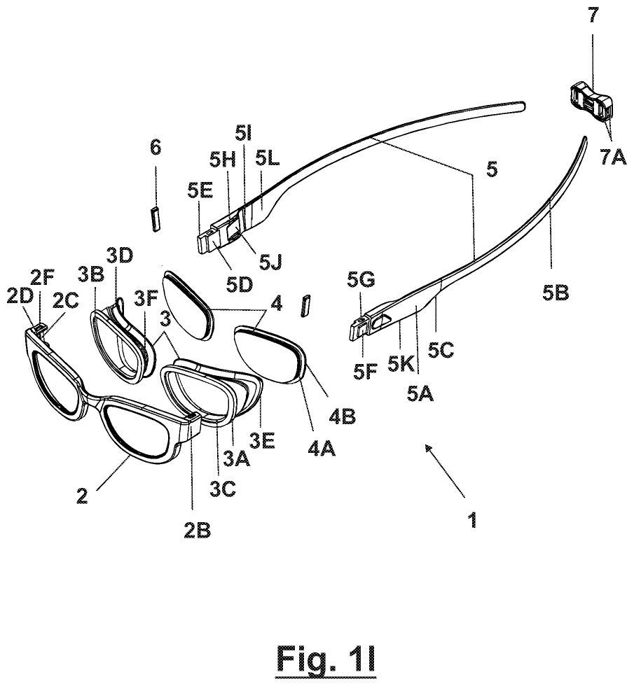

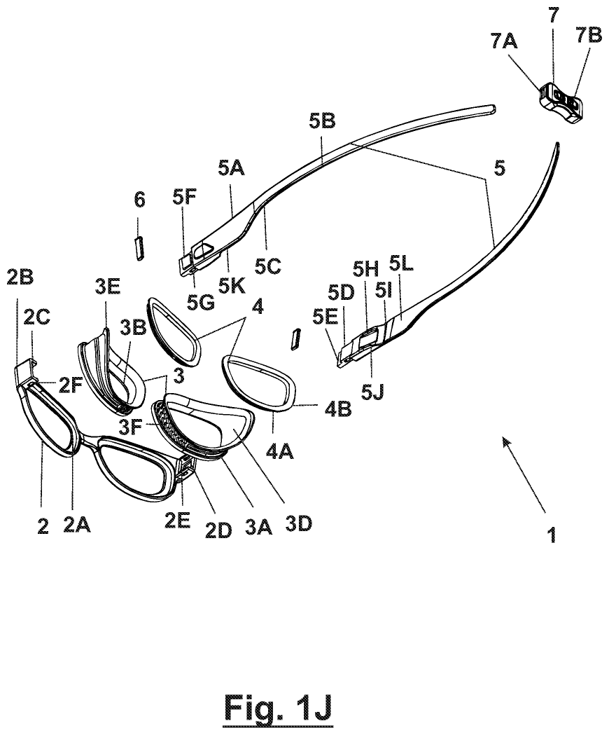

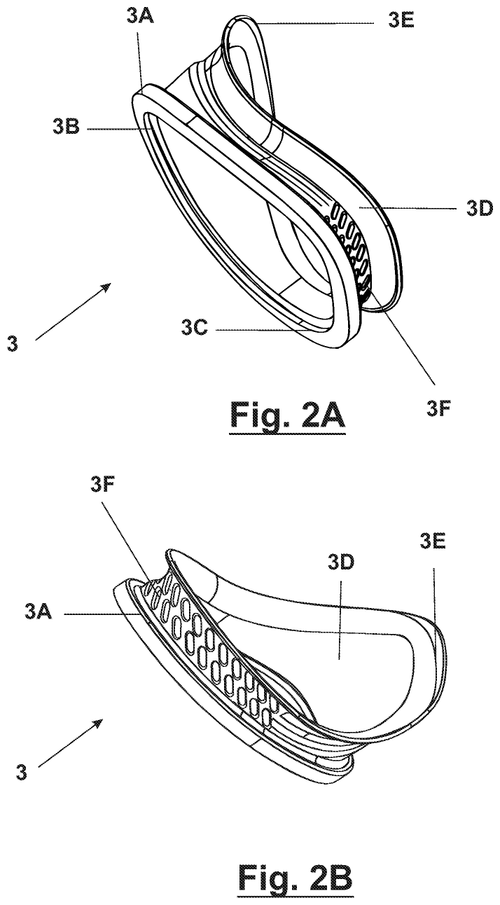

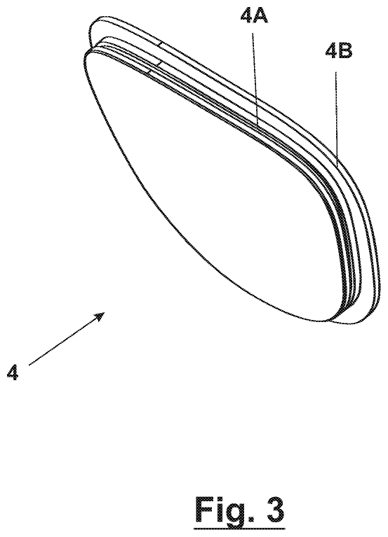

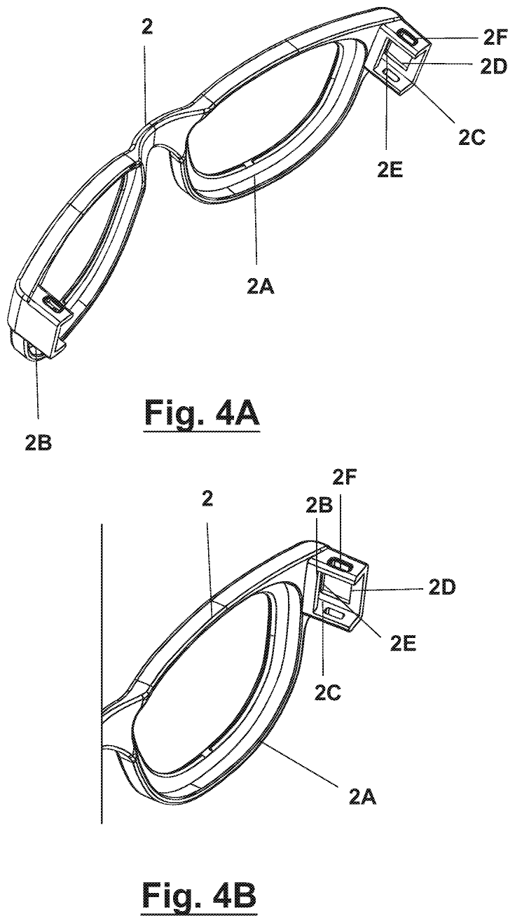

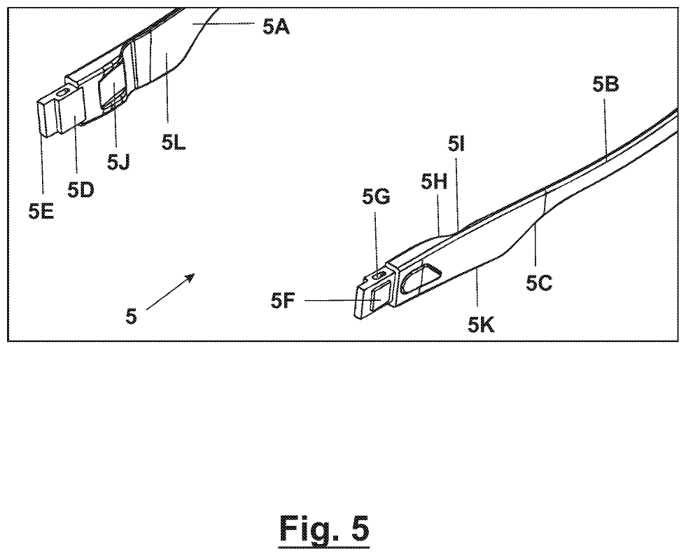

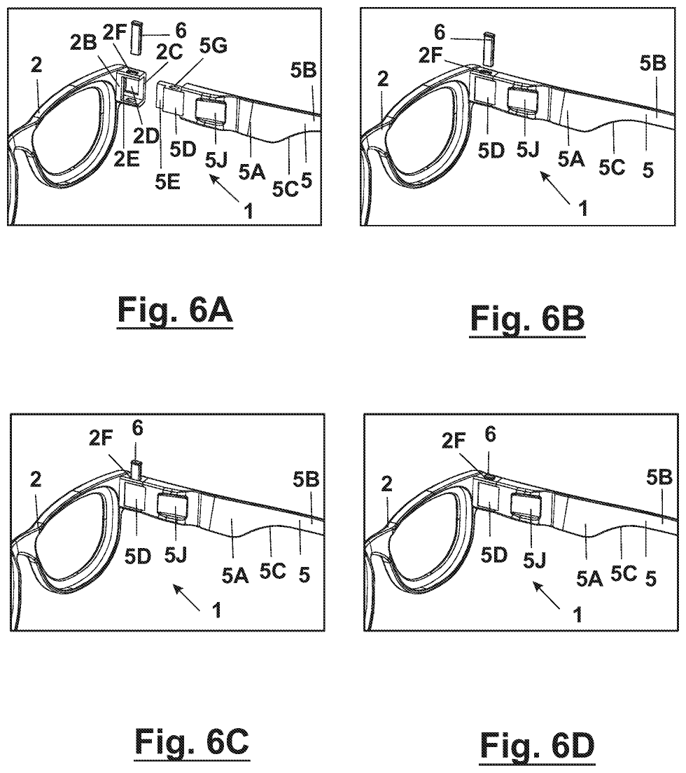

As shown from the accompanying drawings that illustrate and integrate the present descriptive report of the patent of invention “Sunglasses for use in water activities”, FIG. 1A shows the sunglasses for use in water activities in general comprising sunglasses for use in water activities (1) complete and with specific characteristics, incorporating its own specific universal type structure of high durability and resistance, internal and external shapes and arrangements that adapt to the various types of eyes and heads, and containing a properly integrated and symmetrically arranged frame (2) of the contemporary type symmetrically arranged along the entire front length of the sunglasses for use in water activities, with the main function of providing structure for the sunglasses for use in water activities (1); two oblong shaped eye cups (3) symmetrically fixed around the two rear hollow inner portions of the frame (2), with the main function of providing sealing/watertightness and eye area protection in water environments; two oblong shaped lenses (4) symmetrically fitted around the hollow inner portions of the two eye cups (3) by means of a tongue and groove fit, with the main function of providing viewing and protection for the eye area in solar environments (bright light/glare); two curved straps (5) each perpendicular and symmetrically centered by its front ends on the rear side end of the frame (2) by means of a tongue a groove fit, with the main function of securing the glasses around the head; two pins (6), each symmetrically, vertically and transversely fitted in the tongue and groove joint area of the strap (5) and the frame (2), with the main function of holding the straps (5) to the frame (2); and a strap adjuster (7) symmetrically centered on the rear ends of the two straps (5), with the main function of adjusting the glasses around the head.

The single block frame (2) comprises two rear recesses (2A), each parallel and symmetrically arranged along the entire length of the inner side ends of each one of the openings in the frame (2); and two lateral brackets (2B) each perpendicular and symmetrically arranged in a top rear end of the frame (2) and having a lateral cutout (2C) parallel and symmetrically arranged in the inner side of the lateral brackets (2B), a recess (2D) parallel and symmetrically centered on the inner surface of the lateral bracket (2B) and of the lateral cutout (2C), a recess (2E) perpendicular and symmetrically arranged in the front inner part of the lateral cutout (2C), and two openings (2F) symmetrically and vertically aligned between the top and bottom surfaces of the lateral brackets (2B).

Each one of the eye cups (3) is constituted as a single block by a hollow front base (3A), symmetrically arranged along the entire front length of the eye cup (3) to fit the rear recess (2A) of the frame (2) and having a tongue type inner profile (3B) symmetrically arranged along the entire inner length of the front base (3A) and a front recess (3C) symmetrically arranged around the entire length of the front ends of the front base (3A); and a rear bracket (3D) that is pliable, hollow and asymmetrical, perpendicular and symmetrically arranged around the entire length of the rear ends of the front base (3A) and having a rear rim (3E) perpendicular and symmetrically arranged around the entire rear length of the rear bracket (3D), and beehive pattern openings (3F) (non-hollow structure) parallel and symmetrically arranged along the entire outer length of the inner side part of the rear bracket (3D).

Each one of the two lenses (4) is constituted in a single block by a groove type front cutout (4A) perpendicular and symmetrically arranged around the entire length of the outer ends of the lens (4) and fitted in the tongue type inner profile (3B); and a rear rim (4B) perpendicular and symmetrically arranged around the entire length of the rear ends of the lens (2) and pressed into the inner part of the pad (3).

Each one of the two curved straps (5) is constituted in a single block, comprising a thick front body (5A) symmetrically arranged at the front end of the strap (5) in the lateral bracket (2B); and a narrow rear body (5B) symmetrically arranged along the entire length of the strap (5) in the back of the front body (5A) and having a concave curve (5C) symmetrically arranged along the entire bottom length of the intermediate area between the front (5A) and rear (5B) bodies.

The front body (5A) is provided with a front tip (5D) perpendicular and symmetrically centered in the front end of the front body (5A) to be fitted into the lateral cutout (2C), a front projection (5E) perpendicular and symmetrically centered in the front end of the front tip (5D) to be fitted into the recess (2E), an outer projection (5F) parallel and symmetrically centered in the outer surface of the front tip (5D) to be fitted into the recess (2D), a front groove (5G) vertically and symmetrically transversal and centered in the front tip (5D) to be aligned with the two openings (2F), a slight inner convex curve (5H) symmetrically centered in the inner surface of the front body (5A) to allow the strap (5) to bend, a sharp inner concave curve (51) symmetrically arranged in the rear end of the slight inner curve (5H) that allows the strap (5) to bend, an inner recess (5J) parallel and symmetrically centered in the rear part of the inner surface of the front body (5A), a slight bottom tilt (5K) symmetrically arranged along the entire bottom length of the front body (5); and a rear narrowing (5L) parallel and symmetrically arranged along the inner surface of the rear portion of the rear body (5A) and stemming from the sharp inner curve (5I).

Each one of the two pins (6) is constituted in a single block that traverse the two openings (2F), the lateral cutout (2C) and the front groove (5G).

The strap adjuster (7) comprises two anterolateral openings (7A) each symmetrically and internally interconnected between the side ends of the front and side surfaces adjacent to the strap adjuster (7) that traverse the rear end of the rear body (5B); and two snap locks (7B) parallel and symmetrically spaced in the rear outer surface of the strap adjuster (7) and transversally seeping into each anterolateral opening (7A) that fits the rear end of the rear body (5B).

The sunglasses for use in water activities are provided with a simple and direct operation based on the proper placement and handling at the eye area and around users' heads. Care must be taken when securing them around the head, such as the ones described below.

The two eye cups (3) for water environments must be properly positioned/placed and pressed around the eye area, so as to create seamless watertightness in the region, just as the frame (2) must be positioned along the entire front length of the eye area and placed on the nose, and the two lenses (4) for viewing/protection/correction for solar environments placed before the eye area.

The two straps (5) should be slightly flexed along the two sides of the user's head starting at the frame (2) where they are attached by two pins (6) to hold the straps (5) to the frame (2) up to their two rear ends where they are fitted and aligned by the strap adjuster (7).

The fastening of the two straps (5) to the frame (2) is based on a tongue and groove fit between the tongue type front ends of the straps (5) and the groove type rear side ends of the frame (2). Likewise, the fastening of the two straps (5) to the frame (2) is based on the vertical trespassing of the pin (6) into the tongue and groove joint area of the strap (5) and the frame (2).

More specifically, said fastening is carried out by the front body (5A) directly fastened to the lateral bracket (2B), that is, the front tip (5D) jointly fits the lateral cutout (2C), the front projection (5E) fits the recess (2E), the outer projection (5F) fits the recess (2D) and the front groove (5G) is trespassed by the pin (6).

This type of fastening between the two straps (5) and the frame (2) enables, in a simple, practical and safe way, and directly by the users, the combination of different types of frames (2) and straps (5) making use of varied types of designs and colors. Thus, the overall design can be changed as desired by the user at any time.

The innovative design of the two straps (5) is based on a differentiated thickness and geometrical shape along their entire length, starting wider and thicker on their front end, i.e., the area adjacent to the frame (2), and then tapers—becoming thinner and narrower along the entire rear length, so that the thicker front portion is stable or intact and the tapered rear portion dents to fit into the slot, thus ensuring a flawless visual design of the sunglasses for use in water activities (1).

Thus, the design of the two straps (5) is similar to that of traditional/contemporary sunglasses, as well as the comfort and safe fit according to each user's wishes.

More specifically, the assembly formed in the straps (5) by their slight inner curve (5H), sharp inner curve (5I), inner recess (5J), slight bottom tilt (5K) and rear narrowing (5L), as well as by the thick front body (5A) and narrow rear body (5B) with a concave curve (5C) along the entire bottom length of the intermediate area between the front (5A) and rear (5B) bodies, provides high ergonomics and flexibility of the two straps (5) around the head, and also perfect and symmetrical pressure and watertightness of the eye cups (3) around the eye area.

The two eye cups (3) are provided parallelly along the length of the outer part of their inner side surfaces, the surfaces adjacent to the users' nose area, more specifically parallel and symmetrically along the entire outer length of the inner side portion of the rear bracket (3D), a set of beehive pattern openings (3F) that have, together with the material of the two eye cups (3), the main function of creating some degree of support by means of lower flexibility or wear and tear in its structure while being worn around the eye area.

Thus, more comfort and support is achieved when wearing the eye cups (3) by means of a greater support, mainly when adjusting the tightness of the two straps (5) for watertightness without, however, allowing any loss of watertightness and keeping the same design of contemporary sunglasses.

The two viewing/protection lenses (4) for solar environments (bright light) are directly attached and fitted by their outer ends around the inner ends of the two eye cups (3) for water environments by means of a tongue and groove fit, so as to allow seamless watertightness between the elements when in water environments, as well as the same design of the sunglasses for use in water activities.

This type of fastening of the two lenses (4) for eyesight/protection for solar environments (bright light) enables the application, in a simple, practical and safe way, and directly by the users, of different types of eyesight/protection lenses such as different colors, protections and corrections. Thus, the type of correction/protection lenses (4) can be changed, as well as the overall design as desired by the user at any time.

More specifically, this type of fastening of the lens (4) to the pad (3) is carried out by the groove type front profile (4A) that is fitted into the tongue type inner profile (3B), as well as by the rear rim (4B) that is pressure locked into the inner portion of the pad (3).

It should be noted that the fastening of the straps (5) in the strap adjuster (7) is carried out by trespassing the rear ends of the rear bodies (5B) of the straps (5), more specifically by trespassing the rear ends of the rear bodies (5B) through the anterolateral openings (7A) of the front and side surfaces adjacent to the strap adjuster (7), just like the fastening is carried out by engaging the two snap locks (7B) that transversally seep into the anterolateral openings (7A) and press the rear ends of the rear bodies (5B).

As the sunglasses for use in water activities have fully integrated components, it can be assembled and disassembled quickly, nothing comes loose or breaks off, and a high performance and efficiency is achieved, combined with high durability and complete safety during use. Once fully integrated, and adjusted to the wearers' eyes and heads, the components are completely locked and united, thus preventing them from loosening when in use, making the assembly fully available for the helping the user's eyesight and solar and/or water protection. The sunglasses for use in water activities (1) can be thus used without concerns of any nature, mainly regarding the safety and durability of their components, as well as the safety of their users, primarily the eye area.

The sunglasses for use in water activities feature as specific advantages: seamless watertightness of the eye area in water environments; perfect protection of the eye area in situations where bright light/glare is an issue and sun/ultraviolet rays; possibility of eyesight correction due to some kind of visual impairment; possibility of using the most diverse types of combinations/designs in its frame such as social, recreational, sports and professional activities; high versatility while adjusting the glasses around the eye area and the head; and possibility of employing several different types of lenses (4) for eyesight/protection/correction.

In view of the foregoing, the sunglasses for use in water activities will be well received by eye wear users who enjoy swimming and diving in social, recreational or sports activities, since the sunglasses for use in water activities has many advantages, such as: great safety, reliability and flexibility in its application; great efficiency and performance in its application due to its general conception; great comfort, convenience and safety for the users; great overall strength and durability, combined with low or no wear of the assembly as a whole; fully accessible costs which provides an optimum cost-benefit ratio; practical and safe use by any users; high range; perfect and direct adaptation to different types of social, recreational and/or sports applications; great mobility and flexibility of the assembly; high ergonomics; and the certainty of having sunglasses for use in water activities (1) that fully meets the current rules and regulations to its application.

All such attributes classify the sunglasses for use in water activities as a fully versatile, efficient, practical and safe way to be applied in the most diverse types of applications for clear eyesight and solar and/or water protection and several types of users in the most varied types of environments, regardless of the general characteristics these may have. However, it is understood that the present invention is not limited to the specific embodiments described herein, but its objective is to encompass variations and modifications within the scope of protection thereof as defined in the attached claims.

1. Sunglasses for use in water activities, characterized in that said sunglasses for use in water activities (1) incorporates a universal type structure containing integrated and symmetrically arranged:
a frame (2) symmetrically arranged along the entire front length of the sunglasses for use in water activities (1), constituted in a single block by two rear recesses (2A), each parallel and symmetrically arranged around the entire inner side ends of an opening in the frame (2); and two lateral brackets (2B) each perpendicular and symmetrically arranged in a top rear end of the frame (2) and having a side cutout (2C) parallel and symmetrically arranged in the inner portion of the lateral bracket (2B), a recess (2D) parallel and symmetrically centered in the inner surface of the lateral bracket (2B) and the side cutout (2C), a recess (2E) perpendicular and symmetrically arranged in the front inner portion of the front inner portion of the lateral cutout (2C), and two openings (2F) symmetrically and vertically aligned between the top and bottom surfaces of the lateral bracket (2B);
two oblong shaped eye cups (3), symmetrically fixed around the two hollow rear inner portions of the frame (2) each constituting a single block by a hollow front base (3A), symmetrically arranged along the entire front length of the eye cup (3) to fit the rear recess (2A) of the frame (2) and having a tongue type inner profile (3B) symmetrically arranged around the entire inner length of the front base (3A) and a front recess (3C) symmetrically arranged around the entire length of the front ends of the front base (3A); a rear bracket (3D) that is pliable, hollow and asymmetrical, perpendicular and symmetrically arranged around the entire length of the rear ends of the front base (3A), having a rear rim (3E) perpendicular and symmetrically arranged around the entire rear length of the rear bracket (3D), and beehive pattern openings (3F) parallel and symmetrically arranged around the entire outer length of the inner portion of the rear bracket (3D);
two oblong shaped lenses (4) symmetrically fitted around the hollow inner portions of the two eye cups (3) by means of a tongue and groove fit and each constituted in a single block by a groove type front cutout (4A) perpendicular and symmetrically arranged around the entire length of the outer ends of the lens (4) to fit the tongue type inner profile (3B); and a rear rim (4B) perpendicular and symmetrically arranged around the entire length of the rear ends of the lens (2) to press on the inner side of the eye cups (3);
two curved straps (5), each perpendicular and symmetrically centered by its front ends to the rear side end of the frame (2) by means of a tongue and groove fit and constituted in a single block comprising a thick front body (5A) symmetrically arranged in the front end of the strap (5) to fit the lateral bracket (2B); and a narrow rear body (5B) symmetrically arranged along the entire length of the strap (5) in the back of the front body (5A) with a concave curve (5C) symmetrically arranged along the entire bottom length of the intermediate area between the front (5A) and rear (5B) bodies; the front body (5A) having a front tip (5D) perpendicular and symmetrically centered in the front end of the front body (5A) to fit the lateral cutout (2C), a front projection (5E) perpendicular and symmetrically centered in the front end of the front tip (5D) to fit the recess (2E), an outer projection (5F) parallel and symmetrically centered in the outer surface of the front tip (5D) to fit the recess (2D), a front groove (5G) vertically and symmetrically transversal and centered in the front tip (5D) to be aligned with the two openings (2F), a convex inner curve (5H) symmetrically centered in the inner surface of the front body (5A) to make the strap (5) flexible, a concave inner curve (5I) symmetrically arranged in the rear end of the inner curve (5H) to make the strap (5) flexible, an inner recess (5J) parallel and symmetrically centered in the rear portion of the inner surface of the front body (5A), a bottom tilt (5K) symmetrically arranged along the entire bottom length of the front body (5); and a rear narrowing (5L) parallel and symmetrically arranged along the inner surface of the rear portion of the front body (5A) and stemming from the inner curve (5I);
two pins (6), each symmetrically, vertically and transversely fitted in the tongue and groove joint area of the strap (5) and the frame (2) and constituted in a single block that traverse the two openings (2F), the lateral cutout (2C) and the front groove (5G); and
a strap adjuster (7) symmetrically centered on the rear ends of the two straps (5), comprising two anterolateral openings (7A) each symmetrically and internally interconnected between the side ends of the front and side surfaces adjacent to the strap adjuster (7) that traverse the rear end of the rear body (5B); and two snap locks (7B) parallel and symmetrically spaced in the rear outer surface of the strap adjuster (7) and transversally seeping into each anterolateral opening (7A) for the attachment of the rear end of the rear opening (5B).
